SEQUENCE LISTING (10 sequences)

SEQ ID NO: 1 - DNA, 1379 bp, Homo sapiens
  CDS (331)...(1263)
  misc_feature (1)...(1379): n = A,T,C or G
tttggccctcgaggccaagaattcggcacgaggagcaagtggccttaacacatggattttcttccaaaaatgcagacccattttaattaagtttgtaattaaccactggggagggcaggccccctggattcggtctgctttcggagacactgtgagtaacttcctatttgttgaacatttggggattagcacgcccactgggtgttcagcttggaggcttgcacagagctgagctccctgcagccttgggcctccccctgccctgggagtcctgatcagcgtctctttgcaaagccaatccccttttactccgttgtcccccagaacaagatgggagtcatggccatgctgatgctccccctgctgctgctgggaatcagcggcctcctcttcatttaccaagaggtgtccaggctgtggtcaaagtcagctgtgcagaacaaagtggtggtgatcaccgatgccatctcaggactgggcaaggagtgtgctcgggtgttccacacaggtggggcaaggctggtgctgtgtggaaagaactgggagaggctagagaacctatatgatgccttgatcagcgtggctgaccccagcaagacattcaccccaaagctggtcctgttggacctctcagacatcagctgtgtcccagatgtggcaaaagaagtcctggattgctatggctgtgtggacatcctcatcaacaatgccagtgtgaaggtgaaggggcctgcccataagatttctctggagctcgacaaaaagatcatggatgccaattactttggccccatcacattgacgaaagccctgcttcccaacatgatctcccggagaacaggccaaatcgtgttagtgaataatatccaagggaagtttggaatcccgttccgtacgacttacgctgcctccaagcacgcagccctgggcttctttgactgcctccgagccgaagtggaggaatacgatgttgtcatcagcaccgtgagcccgactttcatccggtcgtaccacgtgtatccagagcaaggaaactgggaagcttccatttggaaattctttttcaggaagctgacctacggcgtgcacccagtagaggtggcggaggaggtgatgcgcaccgtgcggaggaagaagcaagaggtgtttatggccaaccccatccccaaggccgccgtgtacgtccgcaccttcttcccggagttctttttcgccgtggtggcctgtggggtgaaggagaagctcaatgtcccggaggaggggtaactgcaggaggccaaatgggccaccccttggaaataaaggtttttctggcaaaaaaaaaaaaaaaaaaaaantttgcggccgcaagcttattccctttagggagggttaatttt

SEQ ID NO: 2 - protein, 311 aa, Homo sapiens
MGVMAMLMLPLLLLGISGLLFIYQEVSRLWSKSAVQNKVVVITDAISGLGKECARVFHTGGARLVLCGKNWERLENLYDALISVADPSKTFTPKLVLLDLSDISCVPDVAKEVLDCYGCVDILINNASVKVKGPAHKISLELDKKIMDANYFGPITLTKALLPNMISRRTGQIVLVNNIQGKFGIPFRTTYAASKHAALGFFDCLRAEVEEYDVVISTVSPTFIRSYHVYPEQGNWEASIWKFFFRKLTYGVHPVEVAEEVMRTVRRKKQEVFMANPIPKAAVYVRTFFPEFFFAVVACGVKEKLNVPEEG

SEQ ID NO: 3 - DNA, 1108 bp, Mus musculus
  CDS (102)...(1034)
ggaatggatgctgttggcttaaacctccccctgccctgggggttgcaaccagggtctctgcaaagccaatcctttgtcatcccgctgtcctgcagagcaagatggggctcatggctgtcctgatgctacccctgctgctgctgggaatcagcggcctcctcttcatttaccaggaggcatccaggctgtggtcgaagtctgccgtgcagaacaaagtggtggtcatcacagatgccatctcaggactgggaaaggagtgtgctcgggtgttccatgcaggtggggcaaggctggtgctgtgtggaaagaactgggagggactggagagcctctatgccaccttgaccagtgtggctgaccccagcaagacattcacccccaagctggtcctcctggatctctcagacattagctgtgttcaagatgtggccaaagaggtcctggactgctacggctgtgtggacatcctcatcaacaatgccagcgtgaaagtgaaggggcctgcccacaagatttccctggagcttgacaaaaagatcatggatgccaactacttcggacccatcactttaaccaaagttctgcttcccaacatgatctccaggagaacaggccagattgtgttagtgaacaacatccaagcgaagtttggaatcccgttccgcacagcttatgcagcctctaagcatgccgtcatgggcttctttgactgcctccgagccgaggttgaggaatacgatgttgtggtcagcaccgtgagcccaactttcatccgctcctaccgtgcttcccctgagcaaagaaactgggagacatccatttgtaaattcttctgcaggaagctagcctatggcgtgcacccggtggaggtggctgaggaagtgatgcgcacagtacggaggaagaagcaagaggtgttcatggccaacccggttcctaaggctgccgtgttcatccgcaccttcttccctgagttcttcttcgctgtggtggcctgtggggtgaaggagaagctcaatgtcccagaagagggttaacctcgtggccaaaggggtcactcaaggggaataaaggctttcctagagaaaaaaaaaaaaaaaaaaaaaaa

SEQ ID NO: 4 - protein, 311 aa, Mus musculus
MGLMAVLMLPLLLLGISGLLFIYQEASRLWSKSAVQNKVVVITDAISGLGKECARVFHAGGARLVLCGKNWEGLESLYATLTSVADPSKTFTPKLVLLDLSDISCVQDVAKEVLDCYGCVDILINNASVKVKGPAHKISLELDKKIMDANYFGPITLTKVLLPNMISRRTGQIVLVNNIQAKFGIPFRTAYAASKHAVMGFFDCLRAEVEEYDVVVSTVSPTFIRSYRASPEQRNWETSICKFFCRKLAYGVHPVEVAEEVMRTVRRKKQEVFMANPVPKAAVFIRTFFPEFFFAVVACGVKEKLNVPEEG

SEQ ID NO: 5 - DNA, 21 bp, Homo sapiens
ccgaagtggaggaatacgatg

SEQ ID NO: 6 - DNA, 20 bp, Homo sapiens
acgtggtacgaccggatgaa

SEQ ID NO: 7 - DNA, 22 bp, Homo sapiens
tcatcagcaccgtgagcccgac

SEQ ID NO: 8 - DNA, 20 bp, Mus musculus
cgaagtttggaatcccgttc

SEQ ID NO: 9 - DNA, 20 bp, Mus musculus
ggcagtcaaagaagcccatg

SEQ ID NO: 10 - DNA, 28 bp, Mus musculus
cacagcttatgcagcctctaagcatgcc

(19) United States
(12) Patent Application Publication
(10) Pub. No.: US 2004/0077566 A9
(43) Pub. Date: Apr. 22, 2004

(54) Methods and compositions for the treatment and diagnosis of body weight disorders

(75) Inventors: Ruth Gimeno, Wellesley, MA (US); Bruce M. Spiegelman, Waban, MA (US)

Correspondence Address: LAHIVE & COCKFIELD, LLP., 28 STATE STREET, BOSTON, MA, 02109, US

(73) Assignee: Millennium Pharmaceuticals, Inc., Cambridge, MA (US)

(21) Appl. No.: 10/144,433

(22) Filed: May 9, 2002

(51) Int. Cl. A61K 48/00; A61K 31/00; A61K 38/17

(52) U.S. Cl. 514/44; 514/12; 514/1

(57) ABSTRACT

The present invention relates to methods and compositions for the treatment and diagnosis of body weight disorders, including, but not limited to, obesity, overweight, anorexia, cachexia, insulin resistance, and diabetes. The invention further provides methods for identifying a compound capable of treating a body weight disorder or modulating thermogenesis. The invention also provides a method for modulating thermogenesis, e.g., modulating thermogenesis in a subject. In addition, the invention provides a method for treating a subject having a body weight disorder characterized by aberrant DHDR-2 polypeptide activity or aberrant DHDR-2 nucleic acid expression. In another aspect, the invention provides methods for modulating thermogenesis in a subject.

### RELATED APPLICATIONS

This application claims the benefit of U.S. Provisional Application Serial No. 60/289,917, filed May 9, 2001, the entire contents of which are incorporated herein by this reference.

### BACKGROUND OF THE INVENTION

Obesity represents the most prevalent of body weight disorders, with estimates ranging from 30% to 50% within the middle-aged population in the western world. Other body weight disorders, such as anorexia nervosa and bulimia nervosa which together affect approximately 0.2% of the female population of the western world, also pose serious health threats. Further body weight disorders such as anorexia and cachexia (wasting) are also prominent features of other diseases such as cancer, cystic fibrosis, and AIDS.

Obesity, defined as a body mass index (BMI) of 30 kg/²m or more, also contributes to other diseases. For example, this disorder is responsible for increased incidences of diseases such as coronary artery disease, hypertension, stroke, diabetes, hyperlipidemia and some cancers (see, e.g., Nishina, P. M. et al. (1994) Metab. 43:554-558; Grundy, S. M. and Barnett, J. P. (1990), Dis. Mon. 36:641-731). Obesity is a complex multifactorial chronic disease that develops from an interaction of genotype and the environment. The development of obesity involves social, behavioral, cultural, physiological, metabolic and genetic factors.

Generally, obesity results when energy intake exceeds energy expenditure. Increasing energy expenditure thus is an important strategy for decreasing body weight. Thermogenesis refers to the ability of the body to convert energy into heat rather than storing it as fat. Thermogenesis occurs mostly in brown adipose tissue (BAT), but is also thought to occur in other tissues, most prominently skeletal muscle. Upon exposing rodents to the cold, a signal transduction cascade is activated in BAT and muscle which culminates in the induction and activation of mitochondrial uncoupling proteins, e.g., UCP1 (expressed exclusively in BAT) and UCP3 (expressed highly and specifically in muscle), key genes involved in effecting thermogenesis. BAT thermogenesis is used both (1) to maintain homeothermy by increasing thermogenesis in response to lower temperatures and (2) to maintain energy balance by increasing energy expenditure in response to increases in caloric intake (Sears, I. B. et al. (1996) Mol. Cell. Biol. 16(7):3410-3419).

A central player in the signal transduction cascade activating thermogenesis is PGC-1. PGC-1 is a transcriptional coactivator which interacts with multiple transcription factors coordinating the expression of groups of genes required for thermogenesis. PGC-1 is primarily expressed in the heart, kidney, BAT, and brain. Upon exposure to the cold, PGC-1 is dramatically upregulated in BAT and muscle, consistent with a role for PGC-1 in thermogenesis in these tissues. Overexpression of PGC-1 in C2C12 muscle cells increases thermogenesis in these cells by increasing both mitochondrial mass and mitochondrial uncoupling, indicating that PGC-1 overexpression is sufficient for inducing thermogenesis in muscle.

Given the prevalence of obesity and other body weight disorders, there currently exists a great need for methods and compositions which can modulate thermogenesis, and which can therefore treat such disorders.

### SUMMARY OF THE INVENTION

The present invention provides methods and compositions for the diagnosis and treatment of body weight disorders. The present invention is based, at least in part, on the discovery that expression of the DHDR-2 gene (for Dehydrogenase-2) is downregulated during cold exposure (i.e., during thermogenesis; see FIG. 1), and is also downregulated in cells expressing the thermogenic coactivator PGC-1 (see FIG. 2). The DHDR-2 molecules, as part of the thermogenic signaling pathway, modulate thermogenesis and are useful as targets and therapeutic agents for the modulation of thermogenesis, e.g., expenditure of energy and the treatment of body weight disorders.

Accordingly, the present invention provides methods for the diagnosis and treatment of disorders or diseases including but not limited to obesity, overweight, anorexia, cachexia, diabetes, and insulin resistance.

In one aspect, the invention provides methods for identifying a compound capable of treating a body weight disorder, e.g., obesity, overweight, anorexia, cachexia, diabetes, and/or insulin resistance. The methods include assaying the ability of the compound to modulate DHDR-2 nucleic acid expression or DHDR-2 polypeptide activity. In one embodiment, the ability of the compound to modulate nucleic acid expression or DHDR-2 polypeptide activity is determined by detecting mitochondrial activity of a cell. In another embodiment, the ability of the compound to modulate nucleic acid expression or DHDR-2 polypeptide activity is determined by detecting modulation of thermogenesis in a cell.

In another aspect, the invention provides methods for identifying a compound capable of modulating thermogenesis. The methods include contacting a cell expressing a DHDR-2 nucleic acid or polypeptide (e.g., a muscle cell such as a primary muscle cell, a C2C12 myocyte, or a C2C12 myotube) with a test compound and assaying the ability of the test compound to modulate the expression of a DHDR-2 nucleic acid or the activity of a DHDR-2 polypeptide.

In a further aspect, the invention features a method for modulating thermogenesis. The method includes contacting a cell (e.g., a muscle cell such as a primary muscle cell, a C2C12 myocyte, or a C2C12 myotube) with a DHDR-2 modulator, for example, an anti-DHDR-2 antibody, a DHDR-2 polypeptide comprising the amino acid sequence of SEQ ID NO:2 or 4, or a fragment thereof, a DHDR-2 polypeptide comprising an amino acid sequence which is at least 90 percent identical to the amino acid sequence of SEQ ID NO:2 or 4, an isolated naturally occurring allelic variant of a polypeptide consisting of the amino acid sequence of SEQ ID NO:2 or 4, a small molecule, an antisense DHDR-2 nucleic acid molecule, a nucleic acid molecule of SEQ ID NO: 1 or 3, or a fragment thereof, or a ribozyme.

In yet another aspect, the invention features a method for treating a subject having a body weight disorder characterized by aberrant DHDR-2 polypeptide activity or aberrant DHDR-2 nucleic acid expression, e.g., obesity, overweight, anorexia, cachexia, diabetes, and/or insulin resistance. The method includes administering to the subject a DHDR-2 modulator, e.g., in a pharmaceutically acceptable formulation or by using a gene therapy vector. In one embodiment, the DHDR-2 modulator may be a small molecule, an anti-DHDR-2 antibody, a DHDR-2 polypeptide comprising the amino acid sequence of SEQ ID NO:2 or 4, or a fragment thereof, a DHDR-2 polypeptide comprising an amino acid sequence which is at least 90 percent identical to the amino acid sequence of SEQ ID NO:2 or 4, an isolated naturally occurring allelic variant of a polypeptide consisting of the amino acid sequence of SEQ ID NO:2 or 4, an antisense DHDR-2 nucleic acid molecule, a nucleic acid molecule of SEQ ID NO: 1 or 3, or a fragment thereof, or a ribozyme.

In another aspect, the invention provides a method for modulating, e.g., increasing or decreasing, thermogenesis in a subject by administering to the subject a DHDR-2 modulator.

Other features and advantages of the invention will be apparent from the following detailed description and claims.

### BRIEF DESCRIPTION OF THE DRAWINGS

FIG. 1 depicts the expression levels of murine DHDR-2 in mixed leg muscle of mice exposed to a temperature of 4° C. for 0 hours, 3 hours, and 24 hours, as determined by Taqman analysis.

FIG. 2 depicts the expression levels of murine DHDR-2 in C2C12 myocytes expressing either GFP (control) or PGC-1, as determined by Taqman analysis.

FIG. 3 depicts the cDNA sequence and predicted amino acid sequence of human DHDR-2 (clone Ep21481, also referred to as clone Fbh21484). The nucleotide sequence corresponds to nucleic acids 1 to 1379 of SEQ ID NO: 1. The amino acid sequence corresponds to amino acids 1 to 311 of SEQ ID NO:2. The coding region is shown as triplets below the amino acid sequence.

FIGS. 4A-4B depict the cDNA sequence and predicted amino acid sequence of mouse DHDR-2 (clone m21481). The nucleotide sequence corresponding to nucleic acids 1 to 1108 of SEQ ID NO:3 is shown in FIG. 4A. The coding region is underlined. The amino acid sequence corresponding to amino acids 1 to 311 of SEQ ID NO:4 is shown in FIG. 4B.

### DETAILED DESCRIPTION OF THE INVENTION

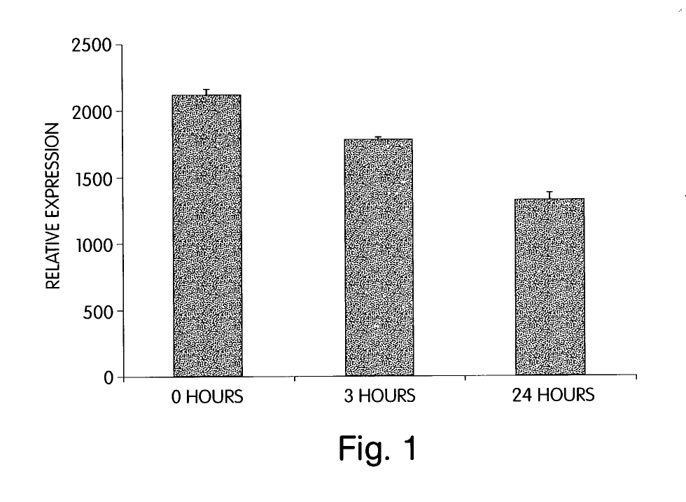

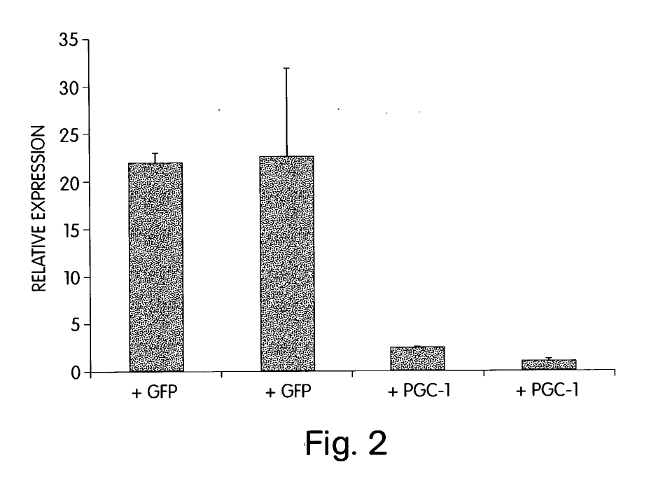

The present invention provides methods and compositions for the diagnosis and treatment of body weight disorders. The present invention is based, at least in part, on the discovery that expression of the DHDR-2 gene (for Dehydrogenase-2) is downregulated during cold exposure (i.e., during thermogenesis; see FIG. 1), and is also downregulated in cells expressing the thermogenic coactivator PGC-1 (see FIG. 2). DHDR-2 is a member of a class of enzymes called short-chain dehydrogenases (SDRs). The SDR family is a large family of enzymes, most of which are known to be NAD/NADP-dependent oxidoreductases. Members of this family include alcohol dehydrogenases, retinol dehydrogenases, steroid dehydrogenases and enzymes involved in FA biosynthesis/elongation (in bacteria and mammals). The closest relative of DHDR-2 is retinal dehydrogenase—members of this family can utilize retinal as well as OH-steroids as substrates to produce retinoic acid as well as steroid derivatives. Accordingly, DHDR-2 may be catalyzing a reaction whose product inhibits thermogenesis. More specifically, given that some members of the SDR family are involved in steroid metabolism, DHDR-2 may be activating or inactivating a ligand for nuclear hormone receptors involved in muscle thermogenesis and/or muscle energy metabolism.

The DHDR-2 modulators identified according to the methods of the invention can be used to modulate thermogenesis, i.e., energy expenditure in, for example, muscle tissue and are, therefore, useful in treating or diagnosing body weight disorders. For example, inhibition of the activity of a DHDR-2 molecule can cause increased thermogenesis and, therefore, increased energy expenditure in a subject, thereby promoting weight loss in the subject. Thus, the DHDR-2 modulators used in the methods of the invention can be used to treat obesity and/or overweight, and/or disorders which are secondary to such disorders. Alternatively, DHDR-2 modulators can decrease thermogenesis by increasing DHDR-2 activity, thus, decreasing energy expenditure in a subject to inhibit weight loss in the subject. Thus, DHDR-2 modulators are also useful in the treatment of undesirable weight loss, e.g., cachexia or anorexia. Modulators of DHDR-2 can also be effective in the treatment of diabetes caused by insulin resistance.

As used herein, a “body weight disorder” includes a disease, disorder, or condition which is associated with abnormal or aberrant body weight. Body weight disorders also include a disease, disorder, or condition associated with aberrant thermogenesis or energy expenditure. Body weight disorders can be characterized by a misregulation (e.g., downregulation or upregulation) of DHDR-2 activity. Examples of body weight disorders include disorders such as obesity, overweight, anorexia, cachexia, and diabetes. Obesity is defined as a body mass index (BMI) of 30 kg/²m or more (National Institute of Health, Clinical Guidelines on the Identification, Evaluation, and Treatment of Overweight and Obesity in Adults (1998)). However, the present invention is also intended to include a disease, disorder, or condition that is characterized by a body mass index (BMI) of 25 kg/²m or more, 26 kg/²m or more, 27 kg/²m or more, 28 kg/²m or more, 29 kg/²m or more, 29.5 kg/²m or more, or 29.9 kg/²m or more, all of which are typically referred to as overweight (National Institute of Health, Clinical Guidelines on the Identification, Evaluation, and Treatment of Overweight and Obesity in Adults (1998)). Body weight disorders also include conditions or disorders which are secondary to disorders such as obesity or overweight, i.e., are influenced or caused by a disorder such as obesity or overweight. For example, insulin resistance, diabetes, hypertension, and atherosclerosis can all be influenced or caused by obesity or overweight. Accordingly, such secondary conditions or disorders are additional examples of body weight disorders, as defined herein.

As used interchangeably herein, “DHDR-2 activity,” “biological activity of DHDR-2” or “functional activity of DHDR-2,” includes an activity exerted by a DHDR-2 protein, polypeptide or nucleic acid molecule on a DHDR-2 responsive cell or tissue (e.g., muscle) or on a DHDR-2 protein substrate, as determined in vivo, or in vitro, according to standard techniques. DHDR-2 activity can be a direct activity, such as an association with a DHDR-2-target molecule. As used herein, a “substrate” or “target molecule” or “binding partner” is a molecule with which a DHDR-2 protein binds or interacts in nature, such that DHDR-2-mediated function, e.g., modulation of thermogenesis, is achieved. A DHDR-2 target molecule can be a non-DHDR-2 molecule (e.g., NAD+, NADP+, or other cofactor, or a biochemical molecule involved in modulating thermogenesis), or a DHDR-2 protein or polypeptide. Examples of such target molecules include proteins in the same signaling path as the DHDR-2 protein, e.g., proteins which may function upstream (including both stimulators and inhibitors of activity) or downstream of the DHDR-2 protein in a pathway involving regulation of thermogenesis. Alternatively, a DHDR-2 activity is an indirect activity, such as a cellular signaling activity mediated by interaction of the DHDR-2 protein with a DHDR-2 target molecule. The biological activities of DHDR-2 are described herein. For example, the DHDR-2 proteins can have one or more of the following activities: 1) they modulate metabolism or catabolism of biochemical molecules (e.g., molecules involved in modulating thermogenesis, such as steroids that may be ligands for nuclear receptors); 2) they modulate thermogenesis (e.g., in muscle); 3) they modulate energy expenditure; and 3) they modulate insulin sensitivity.

Various aspects of the invention are described in further detail in the following subsections:

I. Screening Assays:

The invention provides methods (also referred to herein as “screening assays”) for identifying modulators, i.e., candidate or test compounds or agents (e.g., peptides, peptidomimetics, small molecules, ribozymes, or DHDR-2 antisense molecules) which bind to DHDR-2 proteins, have a stimulatory or inhibitory effect on DHDR-2 expression or DHDR-2 activity, or have a stimulatory or inhibitory effect on the expression or activity of a DHDR-2 target molecule. Compounds identified using the assays described herein may be useful for treating body weight disorders.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one aspect, an assay is a cell-based assay in which a cell which expresses a DHDR-2 protein or biologically active portion thereof is contacted with a test compound and the ability of the test compound to modulate DHDR-2 activity is determined. In a preferred embodiment, the biologically active portion of the DHDR-2 protein includes a domain or motif which can modulate thermogenesis. Determining the ability of the test compound to modulate DHDR-2 activity can be accomplished by monitoring, for example, the production of one or more specific metabolites (e.g., ¹⁴C glucose) or replacement of nutrients in a cell which expresses DHDR-2 (see, e.g., Saada et al. (2000) Biochem. Biophys. Res. Commun. 269:382-386), by measuring expression of thermogenic or mitochondrial genes, or by monitoring mitochondrial content or function in the cell. The cell, for example, can be of mammalian origin, e.g., a muscle cell such as a primary muscle cell, a C2C12 myocyte, or a C2C12 myotube.

The ability of the test compound to modulate DHDR-2 binding to a substrate can also be determined. Determining the ability of the test compound to modulate DHDR-2 binding to a substrate can be accomplished, for example, by coupling the DHDR-2 substrate with a radioisotope, fluorescent, or enzymatic label such that binding of the DHDR-2 substrate to DHDR-2 can be determined by detecting the labeled DHDR-2 substrate in a complex. Alternatively, DHDR-2 could be coupled with a radioisotope or enzymatic label to monitor the ability of a test compound to modulate DHDR-2 binding to a DHDR-2 substrate in a complex. Determining the ability of the test compound to bind DHDR-2 can be accomplished, for example, by coupling the compound with a radioisotope or enzymatic label such that binding of the compound to DHDR-2 can be determined by detecting the labeled DHDR-2 compound in a complex. For example, DHDR-2 substrates can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to interact with DHDR-2 without the labeling of any of the interactants. For example, a microphysiometer can be used to detect the interaction of a compound with DHDR-2 without the labeling of either the-compound or the DHDR-2 (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and DHDR-2.

Because DHDR-2 expression is decreased during thermogenesis, compounds which modulate thermogenesis can be identified by the ability to modulate DHDR-2 expression. To determine whether a test compound modulates DHDR-2 expression, a cell which expresses DHDR-2 (e.g., a muscle cell such as a primary muscle cell, a C2C12 myocyte, or a C2C12 myotube) is contacted with a test compound, and the ability of the test compound to modulate DHDR-2 expression can be determined by measuring DHDR-2 mRNA by, e.g., Northern Blotting, quantitative PCR (e.g., Taqman), or in vitro transcriptional assays. To perform an in vitro transcriptional assay, the full length promoter and enhancer of DHDR-2 can be linked to a reporter gene such as chloramphenicol acetyltransferase (CAT) or luciferase and introduced into host cells. The same host cells can then be transfected with or contacted with the test compound. The effect of the test compound can be measured by reporter gene activity and comparing it to reporter gene activity in cells which do not contain the test compound. An increase or decrease in reporter gene activity indicates a modulation of DHDR-2 expression and is, therefore, an indicator of the ability of the test compound to modulate thermogenesis in muscle cells.

Assays that may be used to identify compounds that modulate DHDR-2 activity also include assays that test for the ability of a compound to modulate thermogenesis. The ability of a test compound to modulate thermogenesis can be measured by its ability to modulate mitochondrial function in a cell which expresses DHDR-2, e.g., a muscle cell such as a primary muscle cell, a C2C12 myocyte, or a C2C12 myotube. For example, the ability of a test compound to modulate thermogenesis can be measured by contacting a cell (e.g., a muscle cell) with the test compound and measuring the number of mitochondria or the level of mitochondrial function in the cell as compared to a control cell not contacted with the test compound. The number of mitochondria can be measured, for example, by counting the mitochondria present in electron microscopy sections of the cell, or by analyzing the amount of mitochondrial DNA present in the cell, for example, by Southern blotting. Mitochondrial function can be determined by measuring expression levels of mitochondrial genes such as cytochrome c oxidase or by measuring oxygen consumption by the cell. The ability of a compound to modulate thermogenesis in the cell can also be measured by measuring the expression level of thermogenic genes (e.g., UCP-3) in the cell. Compounds that modulate thermogenesis can also be identified by performing the above-described assays in animals (e.g., mice) treated to induce thermogenesis. Thermogenesis can be induced in animals by, e.g., exposing them to cold temperatures or treating them with β-adrenergic agents.

Exemplary methods for measuring thermogenesis and mitochondrial function can further be found in: U.S. Pat. No. 6,166,192; PCT International Publication No. WO 00/32215; Puigserver, P. et al. (1998) Cell 92(6):829-39; Vidal-Puig, A. J. et al. (2000) J. Biol. Chem. 275(21):16258-66; and Wu, Z. et al. (1999) Cell 98(1):115-24, the entire contents of all of which are incorporated herein by reference.

The ability of a test compound to modulate insulin sensitivity of a cell can be determined by performing an assay in which cells which express DHDR-2, e.g., muscle cells such as primary muscle cells, C2C12 myocytes or C2C12 myotubes, are contacted with the test compound, e.g., transformed to express the test compound; incubated with radioactively labeled glucose (¹⁴C glucose); and treated with insulin. An increase or decrease in glucose in the cells containing the test compound as compared to control cells indicates that the test compound can modulate insulin sensitivity of the cells. Alternatively, the cells containing the test compound can be incubated with a radioactively labeled phosphate source (e.g., [³²P]ATP) and treated with insulin. Phosphorylation of proteins in the insulin pathway, e.g., the insulin receptor, can then be measured. An increase or decrease in phosphorylation of a protein in the insulin pathway in cells containing the test compound as compared to the control cells indicates that the test compound can modulate insulin sensitivity of the cells.

In yet another embodiment, an assay of the present invention is a cell-free assay in which a DHDR-2 protein or biologically active portion thereof is contacted with a test compound and the ability of the test compound to bind to or to modulate (e.g., stimulate or inhibit) the activity of the DHDR-2 protein or biologically active portion thereof is determined. Preferred biologically active portions of the DHDR-2 proteins to be used in assays of the present invention include fragments which participate in interactions with non-DHDR-2 molecules, e.g., fragments with high surface probability scores. Binding of the test compound to the DHDR-2 protein can be determined either directly or indirectly as described above. Determining the ability of the DHDR-2 protein to bind to a test compound can also be accomplished using a technology such as real-time Biomolecular Interaction Analysis (BIA) (Sjolander, S. and Urbaniczky, C. (1991) Anal Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In yet another embodiment, the cell-free assay involves contacting a DHDR-2 protein or biologically active portion thereof with a known compound which binds the DHDR-2 protein to form an assay mixture, contacting the assay mixture with a test compound, and determining the ability of the test compound to interact with the DHDR-2 protein, wherein determining the ability of the test compound to interact with the DHDR-2 protein comprises determining the ability of the DHDR-2 protein to preferentially bind to or modulate the activity of a DHDR-2 target molecule (e.g., a DHDR-2 substrate).

The cell-free assays of the present invention are amenable to use of both soluble and/or membrane-bound forms of isolated proteins (e.g., DHDR-2 proteins or biologically active portions thereof). In the case of cell-free assays in which a membrane-bound form of an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either DHDR-2 or a DHDR-2 target molecule to facilitate separation of complexed from uncomplexed forms of one or both of the proteins, as well as to accommodate automation of the assay. Binding of a test compound to a DHDR-2 protein, or interaction of a DHDR-2 protein with a DHDR-2 target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/DHDR-2 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or DHDR-2 protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of DHDR-2 binding or activity determined using standard techniques.

Other techniques for immobilizing proteins or cell membrane preparations on matrices can also be used in the screening assays of the invention. For example, either a DHDR-2 protein or a DHDR-2 target molecule can be immobilized utilizing conjugation of biotin and streptavidin. Biotinylated DHDR-2 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with DHDR-2 protein or target molecules but which do not interfere with binding of the DHDR-2 protein to its target molecule can be derivatized to the wells of the plate, and unbound target or DHDR-2 protein is trapped in the wells by antibody conjugation. Methods for detecting such complexes, in addition to those described above for the GST-immobilized complexes, include immunodetection of complexes using antibodies reactive with the DHDR-2 protein or target molecule, as well as enzyme-linked assays which rely on detecting an enzymatic activity associated with the DHDR-2 protein or target molecule.

In yet another aspect of the invention, the DHDR-2 protein or fragments thereof can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300) to identify other proteins which bind to or interact with DHDR-2 (“DHDR-2-binding proteins” or “DHDR-2-bp) and are involved in DHDR-2 activity. Such DHDR-2-binding proteins are also likely to be involved in the propagation of signals by the DHDR-2 proteins or DHDR-2 targets as, for example, downstream elements of a DHDR-2-mediated signaling pathway. Alternatively, such DHDR-2-binding proteins are likely to be DHDR-2 inhibitors.

The two-hybrid system is based on the modular nature of most transcription factors, which consist of separable DNA-binding and activation domains. Briefly, the assay utilizes two different DNA constructs. In one construct, the gene that codes for a DHDR-2 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a DHDR-2-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the DHDR-2 protein.

In another aspect, the invention pertains to a combination of two or more of the assays described herein. For example, a modulating agent can be identified using a cell-based or a cell-free assay, and the ability of the agent to modulate the activity of a DHDR-2 protein can be confirmed in vivo, e.g., in an animal such as an animal model for obesity, diabetes, anorexia, or cachexia. Examples of animals that can be used include the transgenic mouse described in U.S. Pat. No. 5,932,779 that contains a mutation in an endogenous melanocortin-4-receptor (MC4-R) gene; animals having mutations which lead to syndromes that include obesity symptoms (described in, for example, Friedman, J. M. et al. (1991) Mamm. Genome 1:130-144; Friedman, J. M. and Liebel, R. L. (1992) Cell 69:217-220; Bray, G. A. (1992) Prog. Brain Res. 93:333-341; and Bray, G. A. (1989) Amer. J. Clin. Nutr. 5:891-902); the animals described in Stubdal, H. el al. (2000) Mol. Cell Biol. 20(3):878-82 (the mouse tubby phenotype characterized by maturity-onset obesity); the animals described in Abadie, J. M. et al. (2000) Lipids 35(6):613-20 (the obese Zucker rat (ZR), a genetic model of human youth-onset obesity and type 2 diabetes mellitus); the animals described in Shaughnessy, S. et al. (2000) Diabetes 49(6):904-11 (mice null for the adipocyte fatty acid binding protein); or the animals described in Loskutoff, D. J. et al. (2000) Ann. N.Y Acad. Sci. 902:272-81 (the fat mouse). Other examples of animals that may be used include non-recombinant, non-genetic animal models of obesity such as, for example, rabbit, mouse, or rat models in which the animal has been exposed to either prolonged cold or long-term over-eating, thereby, inducing hypertrophy of BAT and increasing BAT thermogenesis (Himms-Hagen, J. (1990) FASEB J. 4(11):2890-8). Additionally, animals created by ablation of BAT through use of targeted expression of a toxin gene (Lowell, B. et al. (1993) Nature 366:740-742) may be used. Animals deficient in PGC-1 (e.g., PGC-1 knockout mice) may be deficient in the ability to induce thermogenesis and therefore may be useful in determining whether a test compound can induce thermogenesis by bypassing PGC-1 and directly modulating the activity of DHDR-2.

Moreover, a DHDR-2 modulator identified as described herein (e.g., an antisense DHDR-2 nucleic acid molecule, a DHDR-2-specific antibody, or a small molecule) can be used in an animal model to determine the efficacy, toxicity, or side effects of treatment with such a modulator. Alternatively, a DHDR-2 modulator identified as described herein can be used in an animal model to determine the mechanism of action of such a modulator.

II. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining DHDR-2 protein and/or nucleic acid expression as well as DHDR-2 activity, in the context of a biological sample (e.g., blood, serum, cells, or tissue, e.g., muscle tissue) to thereby determine whether an individual is afflicted with a body weight disorder. The invention also provides for prognostic (or predictive) assays for determining whether an individual is at risk of developing a body weight disorder. For example, mutations in a DHDR-2 gene can be assayed for in a biological sample. Such assays can be used for prognostic or predictive purpose to thereby prophylactically treat an individual prior to the onset of a body weight disorder.

Another aspect of the invention pertains to monitoring the influence of DHDR-2 modulators (e.g., anti-DHDR-2 antibodies or DHDR-2 ribozymes) on the expression or activity of DHDR-2 in clinical trials.

These and other agents are described in further detail in the following sections.

A. Diagnostic Assays for Body Weight Disorders

To determine whether a subject is afflicted with a body weight disorder, a biological sample may be obtained from a subject and the biological sample may be contacted with a compound or an agent capable of detecting a DHDR-2 protein or nucleic acid (e.g., mRNA or genomic DNA) that encodes a DHDR-2 protein, in the biological sample. A preferred agent for detecting DHDR-2 mRNA or genomic DNA is a labeled nucleic acid probe capable of hybridizing to DHDR-2 mRNA or genomic DNA. The nucleic acid probe can be, for example, the DHDR-2 nucleic acid set forth in SEQ ID NO:I or 3, or a portion thereof, such as an oligonucleotide of at least 15, 20, 25, 30, 25, 40, 45, 50, 100, 250 or 500 nucleotides in length and sufficient to specifically hybridize under stringent conditions to DHDR-2 mRNA or genomic DNA. Other suitable probes for use in the diagnostic assays of the invention are described herein.

A preferred agent for detecting DHDR-2 protein in a sample is an antibody capable of binding to DHDR-2 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of direct substances that can be coupled to an antibody or a nucleic acid probe include various enzymes, prosthetic groups, fluorescent materials, luminescent materials, bioluminescent materials, and radioactive materials. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect DHDR-2 mRNA, protein, or genomic DNA in a biological sample in vitro as well as in vivo. For example, in vitro techniques for detection of DHDR-2 mRNA include Northern hybridizations and in situ hybridizations. In vitro techniques for detection of DHDR-2 protein include enzyme linked immunosorbent assays (ELISAs), Western blots, immunoprecipitations and immunofluorescence. In vitro techniques for detection of DHDR-2 genomic DNA include Southern hybridizations. Furthermore, in vivo techniques for detection of DHDR-2 protein include introducing into a subject a labeled anti-DHDR-2 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting DHDR-2 protein, mRNA, or genomic DNA, such that the presence of DHDR-2 protein, mRNA or genomic DNA is detected in the biological sample, and comparing the presence of DHDR-2 protein, mRNA or genomic DNA in the control sample with the presence of DHDR-2 protein, mRNA or genomic DNA in the test sample.

B. Prognostic Assays for Body Weight Disorder

The present invention further pertains to methods for identifying subjects having or at risk of developing a body weight disorder with aberrant DHDR-2 expression or activity.

As used herein, the term “aberrant” includes a DHDR-2 expression or activity which deviates from the wild type DHDR-2 expression or activity. Aberrant expression or activity includes increased or decreased expression or activity, as well as expression or activity which does not follow the wild type developmental pattern of expression or the subcellular pattern of expression. For example, aberrant DHDR-2 expression or activity is intended to include the cases in which a mutation in the DHDR-2 gene causes the DHDR-2 gene to be under-expressed or over-expressed and situations in which such mutations result in a non-functional DHDR-2 protein or a protein which does not function in a wild-type fashion, e.g., a protein which does not interact with a DHDR-2 substrate, or one which interacts with a non-DHDR-2 substrate.

The assays described herein, such as the preceding diagnostic assays or the following assays, can be used to identify a subject having or at risk of developing a body weight disorder, e.g., obesity, overweight, anorexia, cachexia, insulin resistance, or diabetes. A biological sample may be obtained from a subject and tested for the presence or absence of a genetic alteration. For example, such genetic alterations can be detected by ascertaining the existence of at least one of 1) a deletion of one or more nucleotides from a DHDR-2 gene, 2) an addition of one or more nucleotides to a DHDR-2 gene, 3) a substitution of one or more nucleotides of a DHDR-2 gene, 4) a chromosomal rearrangement of a DHDR-2 gene, 5) an alteration in the level of a messenger RNA transcript of a DHDR-2 gene, 6) aberrant modification of a DHDR-2 gene, such as of the methylation pattern of the genomic DNA, 7) the presence of a non-wild type splicing pattern of a messenger RNA transcript of a DHDR-2 gene, 8) a non-wild type level of a DHDR-2-protein, 9) allelic loss of a DHDR-2 gene, and 10) inappropriate post-translational modification of a DHDR-2-protein.

As described herein, there are a large number of assays known in the art which can be used for detecting genetic alterations in a DHDR-2 gene. For example, a genetic alteration in a DHDR-2 gene may be detected using a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a DHDR-2 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a DHDR-2 gene under conditions such that hybridization and amplification of the DHDR-2 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a DHDR-2 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in DHDR-2 can be identified by hybridizing biological sample derived and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al (1996) Nat. Med. 2:753-759). For example, genetic mutations in DHDR-2 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the DHDR-2 gene in a biological sample and detect mutations by comparing the sequence of the DHDR-2 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the DHDR-2 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type DHDR-2 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in DHDR-2 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a DHDR-2 sequence, e.g., a wild-type DHDR-2 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in DHDR-2 genes. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control DHDR-2 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a DHDR-2 modulator (e.g., an agonist, antagonist, peptidomimetic, protein, peptide, nucleic acid, or small molecule) to effectively treat a body weight.

C. Monitoring of Effects During Clinical Trials

The present invention further provides methods for determining the effectiveness of a DHDR-2 modulator (e.g., a DHDR-2 modulator identified herein) in treating a body weight disorder in a subject. For example, the effectiveness of a DHDR-2 modulator in increasing DHDR-2 gene expression, protein levels, or in upregulating DHDR-2 activity, can be monitored in clinical trials of subjects exhibiting decreased DHDR-2 gene expression, protein levels, or downregulated DHDR-2 activity. Alternatively, the effectiveness of a DHDR-2 modulator in decreasing DHDR-2 gene expression, protein levels, or in downregulating DHDR-2 activity, can be monitored in clinical trials of subjects exhibiting increased DHDR-2 gene expression, protein levels, or DHDR-2 activity. In such clinical trials, the expression or activity of a DHDR-2 gene, and preferably, other genes that have been implicated in, for example, a body weight disorder can be used as a “read out” or marker of the phenotype of a particular cell.

For example, and not by way of limitation, genes, including DHDR-2, that are modulated in cells by treatment with an agent which modulates DHDR-2 activity (e.g., identified in a screening assay as described herein) can be identified. Thus, to study the effect of agents which modulate DHDR-2 activity on subjects suffering from a body weight disorder in, for example, a clinical trial, cells can be isolated and RNA prepared and analyzed for the levels of expression of DHDR-2 and other genes implicated in the body weight disorder. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of DHDR-2 or other genes. In this way, the gene expression pattern can serve as a marker, indicative of the physiological response of the cells to the agent which modulates DHDR-2 activity. This response state may be determined before, and at various points during treatment of the individual with the agent which modulates DHDR-2 activity.

In a preferred embodiment, the present invention provides a method for monitoring the effectiveness of treatment of a subject with an agent which modulates DHDR-2 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a DHDR-2 protein, mRNA, or genomic DNA in the pre-administration sample; (iii) obtaining one or more post-administration samples from the subject; (iv) detecting the level of expression or activity of the DHDR-2 protein, mRNA, or genomic DNA in the post-administration samples; (v) comparing the level of expression or activity of the DHDR-2 protein, mRNA, or genomic DNA in the pre-administration sample with the DHDR-2 protein, mRNA, or genomic DNA in the post administration sample or samples; and (vi) altering the administration of the agent to the subject accordingly. For example, increased administration of the agent may be desirable to increase the expression or activity of DHDR-2 to higher levels than detected, i.e., to increase the effectiveness of the agent. Alternatively, decreased administration of the agent may be desirable to decrease expression or activity of DHDR-2 to lower levels than detected, i.e., to decrease the effectiveness of the agent. According to such an embodiment, DHDR-2 expression or activity may be used as an indicator of the effectiveness of an agent, even in the absence of an observable phenotypic response.

III. Methods of Treatment of Subjects Suffering From Body Weight Disorders:

The present invention provides for both prophylactic and therapeutic methods of treating a subject, e.g., a human, at risk of (or susceptible to) a body weight disorder such as obesity, overweight, anorexia, cachexia, insulin resistance, or diabetes. As used herein, “treatment” of a subject includes the application or administration of a therapeutic agent to a subject, or application or administration of a therapeutic agent to a cell or tissue from a subject, who has a diseases or disorders has a symptom of a disease or disorder, or is at risk of (or susceptible to) a disease or disorder, with the purpose to cure, heal, alleviate, relieve, alter, remedy, ameliorate, improve, or affect the disease or disorder, the symptom of the disease or disorder, or the risk of (or susceptibility to) the disease or disorder. As used herein, a “therapeutic agent” includes, but is not limited to, small molecules, peptides, polypeptides, antibodies, ribozymes, and antisense oligonucleotides.

With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring a subject's prophylactic or therapeutic treatment with either the DHDR-2 molecules of the present invention or DHDR-2 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a body weight disorder by administering to the subject an agent which modulates DHDR-2 expression or DHDR-2 activity, e.g., modulation of thermogenesis in cells, e.g., muscle cells. Subjects at risk for a body weight disorder can be identified by, for example, any or a combination of the diagnostic or prognostic assays described herein. Administration of a prophylactic agent can occur prior to the manifestation of symptoms characteristic of aberrant DHDR-2 expression or activity, such that a body weight disorder is prevented or, alternatively, delayed in its progression. Depending on the type of DHDR-2 aberrancy, for example, a DHDR-2 molecule, DHDR-2 agonist or DHDR-2 antagonist agent can be used for treating the subject. The appropriate agent can be determined based on screening assays described herein.

B. Therapeutic Methods

Another aspect of the invention pertains to methods for treating a subject suffering from a body weight disorder. These methods involve administering to a subject an agent which modulates DHDR-2 expression or activity (e.g., an agent identified by a screening assay described herein), or a combination of such agents. In another embodiment, the method involves administering to a subject a DHDR-2 protein or nucleic acid molecule as therapy to compensate for reduced, aberrant, or unwanted DHDR-2 expression or activity.

Stimulation of DHDR-2 activity is desirable in situations in which DHDR-2 is abnormally downregulated and/or in which increased DHDR-2 activity is likely to have a beneficial effect, i.e., a decrease in thermogenesis, thereby ameliorating a body weight disorder such as anorexia or cachexia in a subject. Likewise, inhibition of DHDR-2 activity is desirable in situations in which DHDR-2 is abnormally upregulated and/or in which decreased DHDR-2 activity is likely to have a beneficial effect, e.g., an increase in thermogenesis, thereby ameliorating a body weight disorder such as obesity, overweight, or diabetes in a subject.

The agents which modulate DHDR-2 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates DHDR-2 activity (e.g., a fragment of a DHDR-2 protein or an anti-DHDR-2 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate DHDR-2 activity can also be prepared in the form of suppositories (e.g., with conventional suppository bases such as cocoa butter and other glycerides) or retention enemas for rectal delivery.

In one embodiment, the agents that modulate DHDR-2 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates DHDR-2 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such DHDR-2 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies for Immunotargeting of Drugs in Cancer Therapy”, in Monoclonal Antibodies and Cancer Therapy, Reisfeld et al. (eds.), pp. 243-56 (Alan R. Liss, Inc. 1985); Hellstrom et al., “Antibodies for Drug Delivery”, in Controlled Drug Delivery (2nd Ed.), Robinson et al. (eds.), pp. 623-53 (Marcel Dekker, Inc. 1987); Thorpe, “Antibody Carriers of Cytotoxic Agents in Cancer Therapy: A Review”, in Monoclonal Antibodies '84: Biological and Clinical Applications, Pinchera et al. (eds.), pp. 475-506 (1985); “Analysis, Results, and Future Prospective of the Therapeutic Use of Radiolabeled Antibody in Cancer Therapy”, in Monoclonal Antibodies for Cancer Detection and Therapy, Baldwin et al. (eds.), pp. 303-16 (Academic Press 1985), and Thorpe et al. (1982) “The Preparation and Cytotoxic Properties of Antibody-Toxin Conjugates”, Immunol. Rev. 62:119-58. Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

C. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates DHDR-2 activity, as well as tailoring the dosage and/or therapeutic regimen of treatment with an agent which modulates DHDR-2 activity.

Pharmacogenomics deals with clinically significant hereditary variations in the response to drugs due to altered drug disposition and abnormal action in affected persons. See, for example, Eichelbaum, M. et al (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a DHDR-2 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a DHDR-2 molecule or DHDR-2 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a body weight disorder with an agent which modulates DHDR-2 activity.

IV. Recombinant Expression Vectors and Host Cells Used in the Methods of the Invention

The methods of the invention (e.g., the screening assays described herein) include the use of vectors, preferably expression vectors, containing a nucleic acid encoding a DHDR-2 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., DHDR-2 proteins, mutant forms of DHDR-2 proteins, fusion proteins, and the like).

The recombinant expression vectors to be used in the methods of the invention can be designed for expression of DHDR-2 proteins in prokaryotic or eukaryotic cells. For example, DHDR-2 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in DHDR-2 activity assays (e.g., direct assays or competitive assays described in detail below), or to generate antibodies specific for DHDR-2 proteins. In a preferred embodiment, a DHDR-2 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to DHDR-2 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a DHDR-2 nucleic acid molecule of the invention is introduced, e.g., a DHDR-2 nucleic acid molecule within a recombinant expression vector or a DHDR-2 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a DHDR-2 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced-into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2^{nd}. ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a DHDR-2 protein. Accordingly, the invention further provides methods for producing a DHDR-2 protein using the host cells of the invention. In one embodiment, the method comprises culturing the host cell of the invention (into which a recombinant expression vector encoding a DHDR-2 protein has been introduced) in a suitable medium such that a DHDR-2 protein is produced. In another embodiment, the method further comprises isolating a DHDR-2 protein from the medium or the host cell.

V. Isolated Nucleic Acid Molecules Used in the Methods of the Invention

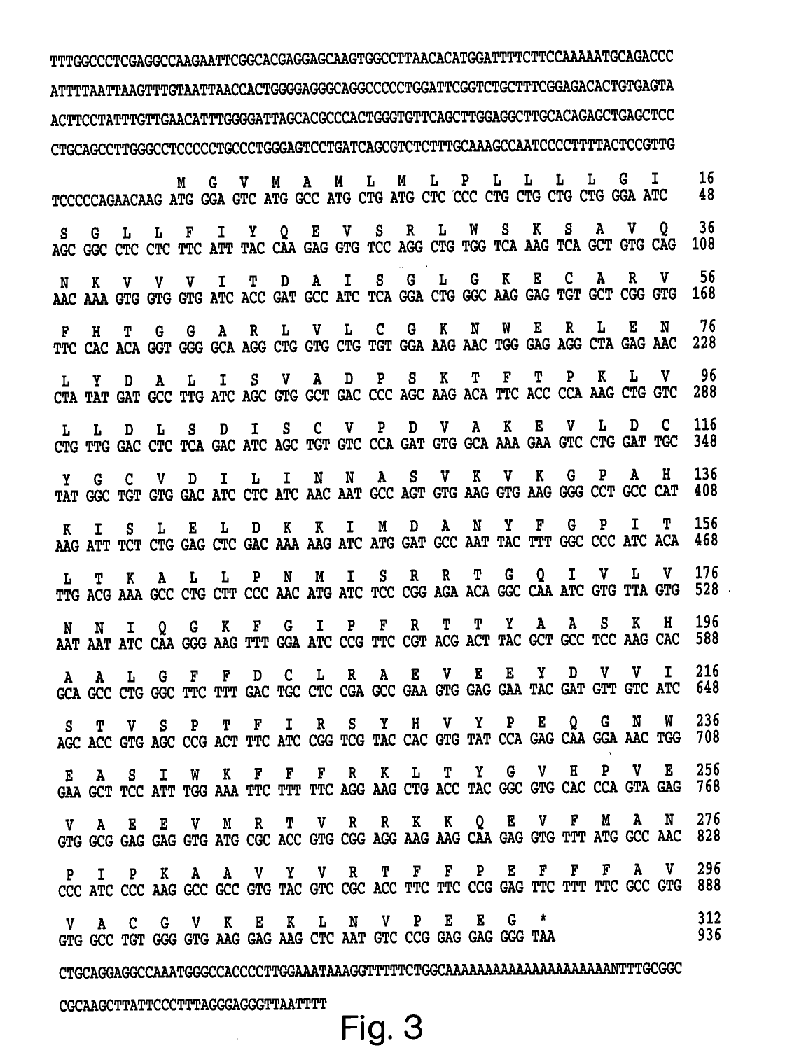

The cDNA sequence of the isolated human DHDR-2 gene and the predicted amino acid sequence of the human DHDR-2 polypeptide are shown in SEQ ID NOs:1 and 2, respectively, and in FIG. 3, and are described in PCT International Publication No. WO 01/72976; as well as in U.S. patent application Ser. No. 09/838,561, filed Apr. 18, 2001; Ser. No. 09/816,760, filed Mar. 23, 2001; Ser. No. 09/634,955, filed Aug. 8, 2000; and U.S. Provisional Application Serial No. 60/192,002, filed Mar. 24, 2000; the entire contents of all of which are incorporated herein by reference. Additionally, a plasmid containing the nucleotide sequence encoding human DHDR-2 was deposited with the American Type Culture Collection (ATCC), 10801 University Boulevard, Manassas, Va. 20110-2209, on May 9, 2000 and assigned Accession Number PTA-1845. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

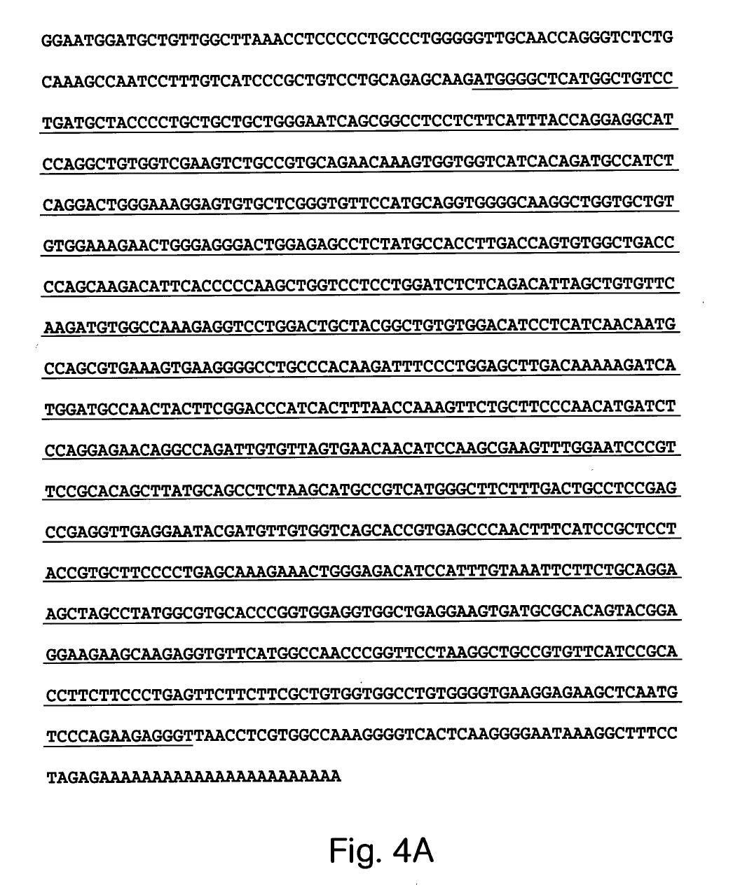

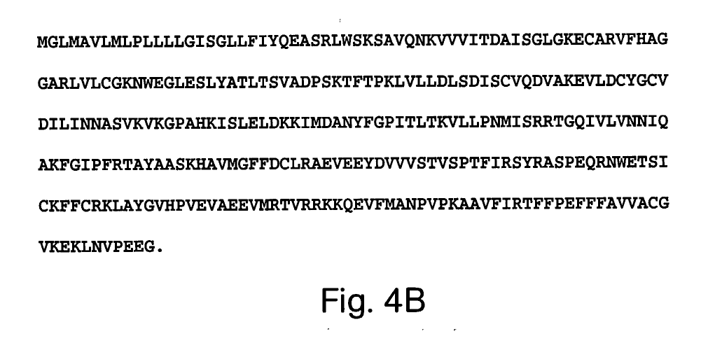

The cDNA sequence of the isolated mouse DHDR-2 gene and the predicted amino acid sequence of the mouse DHDR-2 polypeptide are shown in SEQ ID NOs:3 and 4, and in FIGS. 4A and 4B, respectively, and are described in U.S. patent application Ser. No. 09/838,561, filed Apr. 18, 2001, the entire contents of which are incorporated herein by reference. When aligned using the GAP program in the GCG software package (nwsgapdna.cmp matrix) with a gap weight of 12 and a length weight of 4, the mouse and human DHDR-2 nucleotide sequences are about 88.1% identical. When aligned using the GAP program in the GCG software package (Blosum 62 matrix) with a gap weight of 12 and a length weight of 4, the mouse and human DHDR-2 amino acid sequences are about 91.3% identical.

The methods of the invention include the use of isolated nucleic acid molecules that encode DHDR-2 proteins or biologically active portions thereof, as well as nucleic acid fragments sufficient for use as hybridization probes to identify DHDR-2-encoding nucleic acid molecules (e.g., DHDR-2 mRNA) and fragments for use as PCR primers for the amplification or mutation of DHDR-2 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO: 1 or 3, or a portion thereof, can be isolated using standard molecular biology techniques and the sequence information provided herein. Using all or portion of the nucleic acid sequence of SEQ ID NO: 1 or 3 as a hybridization probe, DHDR-2 nucleic acid molecules can be isolated using standard hybridization and cloning techniques (e.g., as described in Sambrook, J. et al., Molecular Cloning: A Laboratory Manual. 2^{nd}. ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO: 1 or 3 can be isolated by the polymerase chain reaction (PCR) using synthetic oligonucleotide primers designed based upon the sequence of SEQ ID NO: 1 or 3.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to DHDR-2 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO: 1 or 3, a complement of the nucleotide sequence shown in SEQ ID NO: 1 or 3, or a portion of any of these nucleotide sequences. A nucleic acid molecule which is complementary to the nucleotide sequence shown in SEQ ID NO:1 or 3, is one which is sufficiently complementary to the nucleotide sequence shown in SEQ ID NO: 1 or 3 such that it can hybridize to the nucleotide sequence shown in SEQ ID NO: 1 or 3 thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to the entire length of the nucleotide sequence shown in SEQ ID NO: 1 or 3, or a portion of any of this nucleotide sequence.

Moreover, the nucleic acid molecules used in the methods of the invention can comprise only a portion of the nucleic acid sequence of SEQ ID NO: 1 or 3, for example, a fragment which can be used as a probe or primer or a fragment encoding a portion of a DHDR-2 protein, e.g., a biologically active portion of a DHDR-2 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO: 1 or 3 or an anti-sense sequence of SEQ ID NO: 1 or 3, or of a naturally occurring allelic variant or mutant of SEQ ID NO: 1 or 3. In one embodiment, a nucleic acid molecule used in the methods of the present invention comprises a nucleotide sequence which is greater than 50, 50-100, 100-200, 200-300, 300-400, 400-500, 500-600, 600-700, 700-800, 800-900, 900-1000, 1000-1100 or more nucleotides in length and hybridizes under stringent hybridization conditions to a nucleic acid molecule of SEQ ID NO: 1 or 3.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× or 6× sodium chloride/sodium citrate (SSC), at about 65-70° C. (or hybridization in 4×SSC plus 50% formamide at about 42-50° C.) followed by one or more washes in 1×SSC, at about 65-70° C. A further preferred, non-limiting example of stringent hybridization conditions includes hybridization at 6×SSC at 45° C., followed by one or more washes in 0.2×SSC, 0.1% SDS at 65° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4× or 6×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(°C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995 (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a DHDR-2 protein, such as by measuring a level of a DHDR-2-encoding nucleic acid in a sample of cells from a subject e.g., detecting DHDR-2 mRNA levels or determining whether a genomic DHDR-2 gene has been mutated or deleted.

The methods of the invention further encompass the use of nucleic acid molecules that differ from the nucleotide sequence shown in SEQ ID NO: 1 or 3 due to degeneracy of the genetic code and thus encode the same DHDR-2 proteins as those encoded by the nucleotide sequence shown in SEQ ID NO:1 or 3. In another embodiment, an isolated nucleic acid molecule included in the methods of the invention has a nucleotide sequence encoding a protein having an amino acid sequence shown in SEQ ID NO:2 or 4.

The methods of the invention further include the use of allelic variants of human DHDR-2, e.g., functional and non-functional allelic variants. Functional allelic variants are naturally occurring amino acid sequence variants of the human DHDR-2 protein that maintain a DHDR-2 activity. Functional allelic variants will typically contain only conservative substitution of one or more amino acids of SEQ ID NO:2 or 4, or substitution, deletion or insertion of non-critical residues in non-critical regions of the protein. Non-functional allelic variants are naturally occurring amino acid sequence variants of the human DHDR-2 protein that do not have a DHDR-2 activity. Non-functional allelic variants will typically contain a non-conservative substitution, deletion, or insertion or premature truncation of the amino acid sequence of SEQ ID NO:2 or 4, or a substitution, insertion or deletion in critical residues or critical regions of the protein.

The methods of the present invention may further use non-human orthologues of the human DHDR-2 protein. Orthologues of the human DHDR-2 protein are proteins that are isolated from non-human organisms and possess the same DHDR-2 activity.

The methods of the present invention further include the use of nucleic acid molecules comprising the nucleotide sequence of SEQ ID NO: 1 or 3, or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of DHDR-2 (e.g., the sequence of SEQ ID NO:2 or 4) without altering the biological activity, whereas an “essential” amino acid residue is required for biological activity. For example, amino acid residues that are conserved among the DHDR-2 proteins of the present invention and other members of the short-chain dehydrogenase family are not likely to be amenable to alteration.

Mutations can be introduced into SEQ ID NO:1 or 3 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a DHDR-2 protein is preferably replaced with another amino acid residue from the same side chain family. Alternatively, in another embodiment, mutations can be introduced randomly along all or part of a DHDR-2 coding sequence, such as by saturation mutagenesis, and the resultant mutants can be screened for DHDR-2 biological activity to identify mutants that retain activity. Following mutagenesis of SEQ ID NO:1 or 3, the encoded protein can be expressed recombinantly and the activity of the protein can be determined using an assay described herein.

Another aspect of the invention pertains to the use of isolated nucleic acid molecules which are antisense to the nucleotide sequence of SEQ ID NO:1 or 3. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire DHDR-2 coding strand, or to only a portion thereof. In one embodiment, an antisense nucleic acid molecule is antisense to a “coding region” of the coding strand of a nucleotide sequence encoding a DHDR-2. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding DHDR-2. The term “noncoding region” refers to 5′ and 3′ sequences which flank the coding region that are not translated into amino acids (also referred to as 5′ and 3′ untranslated regions).

Given the coding strand sequences encoding DHDR-2 disclosed herein, antisense nucleic acids of the invention can be designed according to the rules of Watson and Crick base pairing. The antisense nucleic acid molecule can be complementary to the entire coding region of DHDR-2 mRNA, but more preferably is an oligonucleotide which is antisense to only a portion of the coding or noncoding region of DHDR-2 mRNA. For example, the antisense oligonucleotide can be complementary to the region surrounding the translation start site of DHDR-2 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules used in the methods of the invention are typically administered to a subject or generated in situ such that they hybridize with or bind to cellular mRNA and/or genomic DNA encoding a DHDR-2 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave DHDR-2 mRNA transcripts to thereby inhibit translation of DHDR-2 mRNA. A ribozyme having specificity for a DHDR-2-encoding nucleic acid can be designed based upon the nucleotide sequence of a DHDR-2 cDNA disclosed herein (i.e., SEQ ID NO: 1 or 3). For example, a derivative of a Tetrahymena L-19 IVS RNA can be constructed in which the nucleotide sequence of the active site is complementary to the nucleotide sequence to be cleaved in a DHDR-2-encoding mRNA. See, e.g., Cech et al., U.S. Pat. No. 4,987,071; and Cech et al., U.S. Pat. No. 5,116,742. Alternatively, DHDR-2 mRNA can be used to select a catalytic RNA having a specific ribonuclease activity from a pool of RNA molecules. See, e.g., Bartel, D. and Szostak, J. W. (1993) Science 261:1411-1418.

Alternatively, DHDR-2 gene expression can be inhibited by targeting nucleotide sequences complementary to the regulatory region of the DHDR-2 (e.g., the DHDR-2 promoter and/or enhancers) to form triple helical structures that prevent transcription of the DHDR-2 gene in target cells. See generally, Helene, C. (1991) Anticancer Drug Des. 6(6):569-84; Helene, C. et al. (1992) Ann. N.Y Acad. Sci. 660:27-36; and Maher, L. J. (1992) Bioessays 14(12):807-15.

In yet another embodiment, the DHDR-2 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup and Nielsen (1996) supra and Perry-O'Keefe et al. (1996) Proc. Natl. Acad. Sci. USA 93:14670-675.

PNAs of DHDR-2 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of DHDR-2 nucleic acid molecules can also be used in the analysis of single base pair mutations in a gene (e.g., by PNA-directed PCR clamping); as ‘artificial restriction enzymes’ when used in combination with other enzymes (e.g., S1 nucleases (Hyrup and Nielsen (1996) supra)); or as probes or primers for DNA sequencing or hybridization (Hyrup and Nielsen (1996) supra; Perry-O'Keefe et al. (1996) supra).

In another embodiment, PNAs of DHDR-2 can be modified (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of DHDR-2 nucleic acid molecules can be generated which may combine the advantageous properties of PNA and DNA. Such chimeras allow DNA recognition enzymes (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup and Nielsen (1996) supra). The synthesis of PNA-DNA chimeras can be performed as described in Hyrup and Nielsen (1996) supra and Finn P. J. et al. (1996) Nucleic Acids Res. 24 (17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acids Res. 17:5973-88). PNA monomers are then coupled in a stepwise manner to produce a chimeric molecule with a 5′ PNA segment and a 3′ DNA segment (Finn et al. (1996) supra). Alternatively, chimeric molecules can be synthesized with a 5′ DNA segment and a 3′ PNA segment (Peterser, K. H. et al. (1975) Bioorganic Med. Chem. Lett. 5:1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (See, e.g., Krol et al. (1988) Biotechniques 6:958-976) or intercalating agents (see, e.g., Zon (1988) Pharm. Res. 5:539-549). To this end, the oligonucleotide may be conjugated to another molecule (e.g., a peptide, hybridization triggered cross-linking agent, transport agent, or hybridization-triggered cleavage agent).

VI. Isolated DHDR-2 Proteins and Anti-DHDR-2 Antibodies Used in the Methods of the Invention

The methods of the invention include the use of isolated DHDR-2 proteins, and biologically active portions thereof, as well as polypeptide fragments suitable for use as immunogens to raise anti-DHDR-2 antibodies. In one embodiment, native DHDR-2 proteins can be isolated from cells or tissue sources by an appropriate purification scheme using standard protein purification techniques. In another embodiment, DHDR-2 proteins are produced by recombinant DNA techniques. Alternative to recombinant expression, a DHDR-2 protein or polypeptide can be synthesized chemically using standard peptide synthesis techniques.

As used herein, a “biologically active portion” of a DHDR-2 protein includes a fragment of a DHDR-2 protein having a DHDR-2 activity. Biologically active portions of a DHDR-2 protein include peptides comprising amino acid sequences sufficiently identical to or derived from the amino acid sequence of the DHDR-2 protein, e.g., the amino acid sequence shown in SEQ ID NO:2 or 4, which include fewer amino acids than the full length DHDR-2 proteins, and exhibit at least one activity of a DHDR-2 protein. Typically, biologically active portions comprise a domain or motif with at least one activity of the DHDR-2 protein. A biologically active portion of a DHDR-2 protein can be a polypeptide which is, for example, 25, 50, 75, 100, 125, 150, 175, 200, 250, 300 or more amino acids in length. Biologically active portions of a DHDR-2 protein can be used as targets for developing agents which modulate a DHDR-2 activity.

In a preferred embodiment, the DHDR-2 protein used in the methods of the invention has an amino acid sequence shown in SEQ ID NO:2 or 4. In other embodiments, the DHDR-2 protein is substantially identical to SEQ ID NO:2 or 4, and retains the functional activity of the protein of SEQ ID NO:2 or 4, yet differs in amino acid sequence due to natural allelic variation or mutagenesis, as described in detail in subsection V above. Accordingly, in another embodiment, the DHDR-2 protein used in the methods of the invention is a protein which comprises an amino acid sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to SEQ ID NO:2 or 4.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the DHDR-2 amino acid sequence of SEQ ID NO:2 or 4 having 311 amino acid residues, at least 93, preferably at least 124, more preferably at least 156, even more preferably at least 187, and even more preferably at least 218, 249, 280 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available online through the Genetics Computer Group), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online through the Genetics Computer Group), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of Meyers, E. and Miller, W. (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use DHDR-2 chimeric or fusion proteins. As used herein, a DHDR-2 “chimeric protein” or “fusion protein” comprises a DHDR-2 polypeptide operatively linked to a non-DHDR-2 polypeptide. A “DHDR-2 polypeptide” refers to a polypeptide having an amino acid sequence corresponding to a DHDR-2 molecule, whereas a “non-DHDR-2 polypeptide” refers to a polypeptide having an amino acid sequence corresponding to a protein which is not substantially homologous to the DHDR-2 protein, e.g., a protein which is different from the DHDR-2 protein and which is derived from the same or a different organism. Within a DHDR-2 fusion protein the DHDR-2 polypeptide can correspond to all or a portion of a DHDR-2 protein. In a preferred embodiment, a DHDR-2 fusion protein comprises at least one biologically active portion of a DHDR-2 protein. In another preferred embodiment, a DHDR-2 fusion protein comprises at least two biologically active portions of a DHDR-2 protein. Within the fusion protein, the term “operatively linked” is intended to indicate that the DHDR-2 polypeptide and the non-DHDR-2 polypeptide are fused in-frame to each other. The non-DHDR-2 polypeptide can be fused to the N-terminus or C-terminus of the DHDR-2 polypeptide.

For example, in one embodiment, the fusion protein is a GST-DHDR-2 fusion protein in which the DHDR-2 sequences are fused to the C-terminus of the GST sequences. Such fusion proteins can facilitate the purification of recombinant DHDR-2.

In another embodiment, this fusion protein is a DHDR-2 protein containing a heterologous signal sequence at its N-terminus. In certain host cells (e.g., mammalian host cells), expression and/or secretion of DHDR-2 can be increased through use of a heterologous signal sequence.

The DHDR-2 fusion proteins used in the methods of the invention can be incorporated into pharmaceutical compositions and administered to a subject in vivo. The DHDR-2 fusion proteins can be used to affect the bioavailability of a DHDR-2 substrate. Use of DHDR-2 fusion proteins may be useful therapeutically for the treatment of disorders caused by, for example, (i) aberrant modification or mutation of a gene encoding a DHDR-2 protein; (ii) mis-regulation of the DHDR-2 gene; and (iii) aberrant post-translational modification of a DHDR-2 protein.

Moreover, the DHDR-2-fusion proteins used in the methods of the invention can be used as immunogens to produce anti-DHDR-2 antibodies in a subject, to purify DHDR-2 ligands and in screening assays to identify molecules which inhibit the interaction of DHDR-2 with a DHDR-2 substrate.

Preferably, a DHDR-2 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al., John Wiley & Sons: 1992). Moreover, many expression vectors are commercially available that already encode a fusion moiety (e.g., a GST polypeptide). A DHDR-2-encoding nucleic acid can be cloned into such an expression vector such that the fusion moiety is linked in-frame to the DHDR-2 protein.

The present invention also pertains to the use of variants of the DHDR-2 proteins which function as either DHDR-2 agonists (mimetics) or as DHDR-2 antagonists. Variants of the DHDR-2 proteins can be generated by mutagenesis, e.g., discrete point mutation or truncation of a DHDR-2 protein. An agonist of the DHDR-2 proteins can retain substantially the same, or a subset, of the biological activities of the naturally occurring form of a DHDR-2 protein. An antagonist of a DHDR-2 protein can inhibit one or more of the activities of the naturally occurring form of the DHDR-2 protein by, for example, competitively modulating a DHDR-2-mediated activity of a DHDR-2 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the DHDR-2 protein.

In one embodiment, variants of a DHDR-2 protein which function as either DHDR-2 agonists (mimetics) or as DHDR-2 antagonists can be identified by screening combinatorial libraries of mutants, e.g., truncation mutants, of a DHDR-2 protein for DHDR-2 protein agonist or antagonist activity. In one embodiment, a variegated library of DHDR-2 variants is generated by combinatorial mutagenesis at the nucleic acid level and is encoded by a variegated gene library. A variegated library of DHDR-2 variants can be produced by, for example, enzymatically ligating a mixture of synthetic oligonucleotides into gene sequences such that a degenerate set of potential DHDR-2 sequences is expressible as individual polypeptides, or alternatively, as a set of larger fusion proteins (e.g., for phage display) containing the set of DHDR-2 sequences therein. There are a variety of methods which can be used to produce libraries of potential DHDR-2 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential DHDR-2 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acids Res. 11:477).

In addition, libraries of fragments of a DHDR-2 protein coding sequence can be used to generate a variegated population of DHDR-2 fragments for screening and subsequent selection of variants of a DHDR-2 protein. In one embodiment, a library of coding sequence fragments can be generated by treating a double stranded PCR fragment of a DHDR-2 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the DHDR-2 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of DHDR-2 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify DHDR-2 variants (Arkin and Youvan (1992) Proc. Natl. Acad. Sci. USA 89:7811-7815; Delagrave et al. (1993) Prot. Eng. 6(3):327-331).

The methods of the present invention further include the use of anti-DHDR-2 antibodies. An isolated DHDR-2 protein, or a portion or fragment thereof, can be used as an immunogen to generate antibodies that bind DHDR-2 using standard techniques for polyclonal and monoclonal antibody preparation. A full-length DHDR-2 protein can be used or, alternatively, antigenic peptide fragments of DHDR-2 can be used as immunogens. The antigenic peptide of DHDR-2 comprises at least 8 amino acid residues of the amino acid sequence shown in SEQ ID NO:2 or 4 and encompasses an epitope of DHDR-2 such that an antibody raised against the peptide forms a specific immune complex with the DHDR-2 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of DHDR-2 that are located on the surface of the protein, e.g., hydrophilic regions, as well as regions with high antigenicity.

A DHDR-2 immunogen is typically used to prepare antibodies by immunizing a suitable subject (e.g., rabbit, goat, mouse, or other mammal) with the immunogen. An appropriate immunogenic preparation can contain, for example, recombinantly expressed DHDR-2 protein or a chemically synthesized DHDR-2 polypeptide. The preparation can further include an adjuvant, such as Freund's complete or incomplete adjuvant, or similar immunostimulatory agent. Immunization of a suitable subject with an immunogenic DHDR-2 preparation induces a polyclonal anti-DHDR-2 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a DHDR-2. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind DHDR-2 molecules. The term “monoclonal antibody” or “monoclonal antibody composition”, as used herein, refers to a population of antibody molecules that contain only one species of an antigen binding site capable of immunoreacting with a particular epitope of DHDR-2. A monoclonal antibody composition thus typically displays a single binding affinity for a particular DHDR-2 protein with which it immunoreacts.

Polyclonal anti-DHDR-2 antibodies can be prepared as described above by immunizing a suitable subject with a DHDR-2 immunogen. The anti-DHDR-2 antibody titer in the immunized subject can be monitored over time by standard techniques, such as with an enzyme linked immunosorbent assay (ELISA) using immobilized DHDR-2. If desired, the antibody molecules directed against DHDR-2 can be isolated from the mammal (e.g., from the blood) and further purified by well known techniques, such as protein A chromatography to obtain the IgG fraction. At an appropriate time after immunization, e.g., when the anti-DHDR-2 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension in Biological Analyses, Plenum Publishing Corp., New York, N.Y. (1980); Lerner, E. A. (1981) Yale J. Biol. Med. 54:387-402; Gefter, M. L. et al. (1977) Somat. Cell Genet. 3:231-36). Briefly, an immortal cell line (typically a myeloma) is fused to lymphocytes (typically splenocytes) from a mammal immunized with a DHDR-2 immunogen as described above, and the culture supernatants of the resulting hybridoma cells are screened to identify a hybridoma producing a monoclonal antibody that binds DHDR-2.

Any of the many well known protocols used for fusing lymphocytes and immortalized cell lines can be applied for the purpose of generating an anti-DHDR-2 monoclonal antibody (see, e.g., Galfre, G. et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind DHDR-2, e.g., using a standard ELISA assay.

Alternative to preparing monoclonal antibody-secreting hybridomas, a monoclonal anti-DHDR-2 antibody can be identified and isolated by screening a recombinant combinatorial immunoglobulin library (e.g., an antibody phage display library) with DHDR-2 to thereby isolate immunoglobulin library members that bind DHDR-2. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al., U.S. Pat. No. 5,223,409; Kang et al., PCT International Publication No. WO 92/18619; Dower et al., PCT International Publication No. WO 91/17271; Winter et al., PCT International Publication No. WO 92/20791; Markland et al., PCT International Publication No. WO 92/15679; Breitling et al., PCT International Publication No. WO 93/01288; McCafferty et al, PCT International Publication No. WO 92/01047; Garrard et al., PCT International Publication No. WO 92/09690; Ladner et al., PCT International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant anti-DHDR-2 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al., International Application No. PCT/US86/02269; Akira et al., European Patent Application No. 184,187; Taniguchi, M., European Patent Application No. 171,496; Morrison et al., European Patent Application No. 173,494; Neuberger et al., PCT International Publication No. WO 86/01533; Cabilly el al., U.S. Pat. No. 4,816,567; Cabilly et al., European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al (1988) Science 239:1534; and Beidler et al. (1988) J. Immunol. 141:4053-4060.

An anti-DHDR-2 antibody can be used to detect DHDR-2 protein (e.g., in a cellular lysate or cell supernatant) in order to evaluate the abundance and pattern of expression of the DHDR-2 protein. Anti-DHDR-2 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, 35S or ³H.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification of DHDR-2 as a Regulator of Muscle Thermogenesis

In order to determine whether the DHDR molecules of the present invention are involved in muscle thermogenesis, gene expression in the mixed leg muscle of mice housed at room temperature was compared to gene expression in the mixed leg muscle of mice housed at 4° C. for varying amounts of time. Regulation of DHDR expression by the thermogenic coactivator PGC-1 was also examined in C2C12 muscle cells.

Materials and Methods

For analysis of human and murine DHDR-2 expression during thermogenesis, the following methods were used:

Tissues were collected from 7 week old female C57/B16J mice (control panel) and 6 week old male C57/B16J mice. Total RNA was prepared using the trizol method and treated with DNAse to remove contaminating genomic DNA. cDNA was synthesized using standard techniques. Mock cDNA synthesis in the absence of reverse transcriptase resulted in samples with no detectable PCR amplification of the control 18S RNA gene confirming efficient removal of genomic DNA contamination. DHDR-2 expression was measured by TaqMan® quantitative PCR analysis, performed according to the manufacturer's directions (Perkin Elmer Applied Biosystems, Foster City, Calif.).

The tissue samples included the following normal mouse tissues: BAT (brown adipose tissue), WAT (white adipose tissue), brain-hypothalamus, hypothalamus, skeletal muscle, liver, kidney, heart, intestine, and spleen.

PCR probes were designed by PrimerExpress software (PE Biosystems) based on the sequence of human DHDR-2 (SEQ ID NO:1) and murine DHDR-2 (SEQ ID NO:3). The following probes and primers were used: human DHDR-2 forward primer: 5′ CCG AAG TGG AGG AAT ACG ATG 3′ (SEQ ID NO:5); human DHDR-2 reverse primer: 5′ ACG TGG TAC GAC CGG ATG AA 3′ (SEQ ID NO:6); human DHDR-2 probe: 5′ TCA TCA GCA CCG TGA GCC CGA C 3′ (SEQ ID NO:7); murine DHDR-2 forward primer: 5′ CGA AGT TTG GAA TCC CGT TC 3′ (SEQ ID NO:8); murine DHDR-2 reverse primer: 5′ GGC AGT CAA AGA AGC CCA TG 3′ (SEQ ID NO:9); and murine DHDR-2 probe: 5′ CAC AGC TTA TGC AGC CTC TAA GCA TGC C 3′ (SEQ ID NO:10).

To standardize the results between different tissues, two probes, distinguished by different fluorescent labels, were added to each sample. The differential labeling of the probe for the DHDR-2 gene and the probe for 18S RNA as an internal control thus enabled their simultaneous measurement in the same well. Forward and reverse primers and the probes for both 18S RNA and human or murine DHDR-2 were added to the TaqMan Universal PCR Master Mix (PE Applied Biosystems). Although the final concentration of primer and probe could vary, each was internally consistent within a given experiment. A typical experiment contained 200 nM of forward and reverse primers, plus 100 nM of the probe for the 18S RNA, and 4500 nM of each of the forward and reverse primers, plus 150 nM of the probe for murine DHDR-2. TaqMan matrix experiments were carried out using an ABI PRISM 770 Sequence Detection System (PE Applied Biosystems). The thermal cycler conditions were as follows: hold for 2 minutes at 50° C. and 10 minutes at 95° C., followed by two-step PCR for 40 cycles of 95° C. for 15 seconds, followed by 60° C. for 1 minute.

The following method was used to quantitatively calculate murine DHDR-2 gene expression in the tissue samples, relative to the 18S RNA expression in the same tissue. The threshold values at which the PCR amplification started were determined using the manufacturer's software. PCR cycle number at threshold value was designated as CT. Relative expression was calculated as:

2⁻⁽⁽CTtest−CT18S)tissue of interest−(CTtest−CT18S)lowest expressing tissue in panel)

Samples were run in duplicate and the averages of 2 relative expression determinations are shown. All probes were tested on serial dilutions of RNA from a tissue with high expression levels and only probes which gave relative expression levels that were linear to the amount of template cDNA with a slope similar to the slope for the internal control 18S were used.

For Northern Blotting, human mRNA blots (Clontech) were probed with a 520 nucleotide SacI fragment containing 420 nucleotides of the 5′ coding sequence and 100 nucleotides of the 5′ UTR of human DHDR-2 (SEQ ID NO: 1). Probes were labeled with ³²p and hybridized using the Rapid-Hyb buffer (Amersham).

Results

The expression of DHDR-2 was examined in a variety of normal mouse and human tissues using Taqman analysis. The results indicated that DHDR-2 was most highly expressed in skeletal muscle and was also present in high levels in the heart, but was absent from a large number of other tissues, including smooth muscle tissues. To verify expression of DHDR-2 in skeletal muscle, Northern Blotting was performed using commercially available Clontech Blots. A single mRNA species of ˜1.35 kD was detected, which is in agreement with the size of the cloned cDNA (1.378 kD). DHDR-2 was most highly expressed in skeletal muscle, was present in heart at considerable levels and was not detectable in other tissues, confirming the expression data determined by TaqMan analysis.

To demonstrate regulation of DHDR-2 in muscle in the cold, TaqMan analysis was performed on RNA from mixed leg muscle of mice exposed to 4° C. for 0, 3 or 24 hours. The results indicated that DHDR-2 expression decreased with increasing exposure to cold (FIG. 1). DHDR-2 expression was also examined in C2C12 myocytes infected with adenovirus expressing either GFP or PGC-1. DHDR-2 expression was more than 5-fold lower in PGC-1 infected cells as compared to the controls (FIG. 2). The down-regulated expression of DHDR-2 in these models of thermogenesis indicates a role for DHDR-2 in thermogenesis in the muscle.

The results described above demonstrate that DHDR-2 is a relatively muscle-specific gene that is regulated during muscle thermogenesis. DHDR-2 expression in the muscle may be blocking thermogenesis and, accordingly, inhibitors of DHDR-2 would stimulate thermogenesis and, thus, be useful as agents which can increase energy expenditure, and therefore, treat body weight disorders.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a compound capable of treating a body weight disorder comprising assaying the ability of the compound to modulate DHDR-2 nucleic acid expression or DHDR-2 polypeptide activity, thereby identifying a compound capable of treating a body weight disorder.

2. The method of claim 1, wherein the body weight disorder is selected from the group consisting of obesity, overweight, diabetes, insulin resistance, cachexia, and anorexia.

3. The method of claim 1, wherein the ability of the compound to modulate DHDR-2 nucleic acid expression or DHDR-2 polypeptide activity is determined by detecting mitochondrial activity of a cell.

4. The method of claim 1, wherein the ability of the compound to modulate DHDR-2 nucleic acid expression or DHDR-2 polypeptide activity is determined by detecting thermogenesis in a cell.

5. A method for identifying a compound capable of modulating thermogenesis comprising:
a) contacting a cell which expresses DHDR-2 with a test compound; and
b) assaying the ability of the test compound to modulate the expression of a DHDR-2 nucleic acid or the activity of a DHDR-2 polypeptide, thereby identifying a compound capable of modulating a thermogenesis.

6. A method for modulating thermogenesis in a cell comprising contacting a cell with a DHDR-2 modulator, thereby modulating thermogenesis in the cell.

7. The method of any one of claims 5 or 6, wherein the cell is a muscle cell.

8. The method of claim 7, wherein the muscle cell is selected from the group consisting of a primary muscle cell, a C2C12 myocyte, and a C2C12 myotube.

9. The method of claim 6, wherein the DHDR-2 modulator is a small molecule.

10. The method of claim 6, wherein the DHDR-2 modulator is capable of modulating DHDR-2 polypeptide activity.

11. The method of claim 10, wherein the DHDR-2 modulator is an anti-DHDR-2 antibody.

12. The method of claim 10, wherein the DHDR-2 modulator is a DHDR-2 polypeptide comprising the amino acid sequence of SEQ ID NO:2 or 4, or a fragment thereof.

13. The method of claim 10, wherein the DHDR-2 modulator is a DHDR-2 polypeptide comprising an amino acid sequence which is at least 90 percent identical to the amino acid sequence of SEQ ID NO:2 or 4, wherein said percent identity is calculated using the ALIGN program for comparing amino acid sequences, a PAM 120 weight residue table, a gap length penalty of 12, and a gap penalty of 4.

14. The method of claim 10, wherein the DHDR-2 modulator is an isolated naturally occurring allelic variant of a polypeptide consisting of the amino acid sequence of SEQ ID NO:2 or 4, wherein the polypeptide is encoded by a nucleic acid molecule which hybridizes to a complement of a nucleic acid molecule consisting of SEQ ID NO:1 or 3 at 6×SSC at 45° C., followed by one or more washes in 0.2×SSC, 0.1% SDS at 65° C.

15. The method of claim 6, wherein the DHDR-2 modulator is capable of modulating DHDR-2 nucleic acid expression.

16. The method of claim 15, wherein the DHDR-2 modulator is an antisense DHDR-2 nucleic acid molecule.

17. The method of claim 15, wherein the DHDR-2 modulator is a ribozyme.

18. The method of claim 15, wherein the DHDR-2 modulator comprises the nucleotide sequence of SEQ ID NO:1 or 3, or a fragment thereof.

19. A method for treating a subject having a body weight disorder characterized by aberrant DHDR-2 polypeptide activity or aberrant DHDR-2 nucleic acid expression comprising administering to the subject a DHDR-2 modulator, thereby treating said subject having a body weight disorder.

20. The method of claim 19, wherein said body weight disorder is selected from the group consisting of obesity, overweight, diabetes, insulin resistance, cachexia, and anorexia.

21. The method of claim 19, wherein said DHDR-2 modulator is administered in a pharmaceutically acceptable formulation.

22. The method of claim 19, wherein said DHDR-2 modulator is administered using a gene therapy vector.

23. The method of claim 19, wherein the DHDR-2 modulator is a small molecule.

24. The method of claim 19, wherein the DHDR-2 modulator is capable of modulating DHDR-2 polypeptide activity.

25. The method of claim 24, wherein the DHDR-2 modulator is an anti-DHDR-2 antibody.

26. The method of claim 24, wherein the DHDR-2 modulator is a DHDR-2 polypeptide comprising the amino acid sequence of SEQ ID NO:2 or 4, or a fragment thereof.

27. The method of claim 24, wherein the DHDR-2 modulator is a DHDR-2 polypeptide comprising an amino acid sequence which is at least 90 percent identical to the amino acid sequence of SEQ ID NO:2 or 4, wherein said percent identity is calculated using the ALIGN program for comparing amino acid sequences, a PAM 120 weight residue table, a gap length penalty of 12, and a gap penalty of 4.

28. The method of claim 24, wherein the DHDR-2 modulator is an isolated naturally occurring allelic variant of a polypeptide consisting of the amino acid sequence of SEQ ID NO:2 or 4, wherein the polypeptide is encoded by a nucleic acid molecule which hybridizes to a complement of a nucleic acid molecule consisting of SEQ ID NO: 1 or 3 at 6×SSC at 45° C., followed by one or more washes in 0.2×SSC, 0.1% SDS at 65° C.

29. The method of claim 19, wherein the DHDR-2 modulator is capable of modulating DHDR-2 nucleic acid expression.

30. The method of claim 29, wherein the DHDR-2 modulator is an antisense DHDR-2 nucleic acid molecule.

31. The method of claim 29, wherein the DHDR-2 modulator is a ribozyme.

32. The method of claim 29, wherein the DHDR-2 modulator comprises the nucleotide sequence of SEQ ID NO: 1 or 3, or a fragment thereof.

33. A method for modulating thermogenesis in a subject comprising administering to the subject a DHDR-2 modulator, thereby modulating thermogenesis in said subject.
